(19) United States
(12) Patent Application Publication
(10) Pub. No.: US 2025/0073420 A1
(43) Pub. Date: Mar. 6, 2025

(54) CONTROLLED DESICCANT ABSORPTION AND POSITIONING SYSTEM FOR MEDICAL DEVICES

(71) Applicant: Boston Scientific Scimed, Inc., Maple Grove, MN (US)

(72) Inventors: Frank Haven Matti, Shoreview, MN (US); Robert Boyd Ewart, Mahtomedi, MN (US); Daniel Burgess, Duluth, MN (US); Mark Harding, Minnetrista, MN (US)

(73) Assignee: Boston Scientific Scimed, Inc., Maple Grove, MN (US)

(21) Appl. No.: 18/812,265

(22) Filed: Aug. 22, 2024

Related U.S. Application Data
(60) Provisional application No. 63/535,499, filed on Aug. 30, 2023.

(51) Int. Cl. A61M 25/00; A61B 50/30

(52) U.S. Cl. CPC A61M 25/002; A61B 2050/314

(57) ABSTRACT

Medical device packaging, medical device packaging assemblies, and methods for preparing and packaging medical devices. An example medical device packaging assembly may include an external package, a carrier tube disposed within the external package, the carrier tube including one or more clips configured to hold the carrier tube in a predetermined configuration, a medical device disposed within the carrier tube, and a moisture or oxygen scavenging packet releasably coupled to one of the one or more clips. The moisture or oxygen scavenging packet may comprise an outer barrier pouch, an inner container disposed within the outer barrier pouch, and a desiccant and/or an oxygen scavenging material disposed within the inner container.

### CROSS REFERENCE TO RELATED APPLICATIONS

This application claims the benefit of priority of U.S. Provisional Application No. 63/535,499, filed Aug. 30, 2023, the entire disclosure of which is hereby incorporated by reference.

### TECHNICAL FIELD

The present disclosure pertains to medical devices, medical device packaging, and methods for preparing and packaging medical devices. More particularly, the present disclosure pertains to medical device packages that include a single pouch, barrier package, tray, or other package.

### BACKGROUND

A wide variety of intracorporeal medical devices have been developed for medical use, for example, intravascular use. Some of these devices include guidewires, catheters, and the like. These devices are manufactured, packaged, and/or prepared by any one of a variety of methods. Of the known medical devices and methods, each has certain advantages and disadvantages. There is an ongoing need to provide alternative medical devices as well as alternative methods for manufacturing, packaging, and/or preparing medical devices.

### BRIEF SUMMARY

Embodiments of the present disclosure provide design, material, and use alternatives for medical device packaging and packaging assemblies, as well as methods for preparing and packaging medical devices.

In a first example, a medical device packaging assembly may comprise a packaging pouch including an external package, a carrier tube disposed within the external package, the carrier tube including one or more clips configured to hold the carrier tube in a predetermined configuration, a medical device disposed within the carrier tube, and a moisture or oxygen scavenging packet releasably coupled to one of the one or more clips. The moisture or oxygen scavenging packet may comprise an outer barrier pouch, an inner container disposed within the outer barrier pouch, and a desiccant and/or an oxygen scavenging material disposed within the inner container.

Alternatively or additionally to any of the examples above, in another example, the inner container may comprise a non-porous material.

Alternatively or additionally to any of the examples above, in another example, the outer barrier pouch may comprise a porous material.

Alternatively or additionally to any of the examples above, in another example, the inner container may be configured to rupture in response to an applied force.

Alternatively or additionally to any of the examples above, in another example, the inner container may comprise a sachet formed from a polymer film.

Alternatively or additionally to any of the examples above, in another example, the inner container may comprise a crushable vial.

Alternatively or additionally to any of the examples above, in another example, the outer barrier pouch may comprise a sealed header region.

Alternatively or additionally to any of the examples above, in another example, the medical device packaging assembly may further comprise an opening extending through a thickness of the sealed header region.

In another example, a method for packaging a medical device may comprise coupling an environmental control packet to a clip secured relative to a medical device. The environmental control packet may comprise an outer barrier pouch, an inner container disposed within the outer barrier pouch, and a desiccant and/or an oxygen scavenging material disposed within the inner container. The method may further comprise disposing the medical device, clip, and environmental control packet within an external package, sealing the external package, sterilizing the external package, rupturing the inner container, and gas flushing the external package.

Alternatively or additionally to any of the examples above, in another example, rupturing the inner container and gas flushing the external package may occur substantially simultaneously.

Alternatively or additionally to any of the examples above, in another example, the method may further comprise positioning a restrictor plate over the environmental control packet prior to gas flushing the external package.

Alternatively or additionally to any of the examples above, in another example, rupturing the inner container may comprise applying a physical force to the inner container.

Alternatively or additionally to any of the examples above, in another example, rupturing the inner container may comprise applying a pressure differential to the inner container.

Alternatively or additionally to any of the examples above, in another example, rupturing the inner container may comprise exposing the inner container to a predetermined temperature.

Alternatively or additionally to any of the examples above, in another example, rupturing the inner container may activate a useful life of the desiccant and/or oxygen scavenging material.

In another example, an environmental control packet for use with a medical device packaging assembly may comprise a porous outer barrier pouch, a non-porous inner container disposed within the outer barrier pouch, and a desiccant and/or an oxygen scavenging material disposed within the inner container. The inner container may be configured to rupture in response to an applied force.

Alternatively or additionally to any of the examples above, in another example, the inner container may comprise a sachet formed from a polymer film.

Alternatively or additionally to any of the examples above, in another example, the inner container may comprise a crushable vial.

Alternatively or additionally to any of the examples above, in another example, the outer barrier pouch may comprise a sealed header region.

Alternatively or additionally to any of the examples above, in another example, the environmental control packet may further comprise an opening extending through a thickness of the sealed header region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

Embodiments of the present disclosure may be more completely understood in consideration of the following detailed description in connection with the accompanying drawings, in which:

FIG. 1 is a side view of an example medical device disposed within a carrier tube;

FIG. 2 is a side view of the example medical device shown in FIG. 1 disposed within an example medical device package;

FIG. 3 is a schematic top view of an illustrative packet for enclosing a desiccant or oxygen scavenging material;

FIG. 4 is a partial cross-sectional view of the illustrative packet of FIG. 3, taken at line 4-4 of FIG. 3;

FIG. 5 is a schematic perspective view of the packet coupled with the label retention clip;

FIG. 6 is an illustrative flow chart of a method for packaging the medical device to from a packaging assembly and activating that the desiccant;

FIG. 7 is a schematic cross-sectional view of the packet during the sterilization process;

FIG. 8 is a schematic cross-sectional view of the packet during the gas flushing process with a restrictor plate; and

FIG. 9 is a schematic cross-sectional view of the packet after the gas flushing process and/or after rupture of the inner sachet.

While embodiments described herein are amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

For obvious reasons, medical devices are typically sterilized or otherwise prepared for use prior to sale. In order to preserve sterility, medical devices are usually contained within appropriate packaging that allows the sterile medical devices to be shipped to and stored by the purchaser. For example, prior to sale, medical devices such as, but not limited to, catheters are typically sterilized and packaged. When suitably prepared, the catheters can be transported to an appropriate medical setting where they can be stored until needed for use. While the present disclosure is described with respect to catheters, the packaging system and the moisture and/or oxygen scavenging packets/sachets may be used with other medical devices, or sterilized devices, as desired.

Once packaged, it may be desirable to limit the exposure of a number of medical devices to particular substances and/or conditions. For example, it may be desirable to control or limit the medical device's exposure to moisture and/or oxygen. This may be particularly relevant when the medical device includes a material that may be adversely affected by exposure to oxygen and/or moisture. For example, some medical devices may include a drug-coated stent that can swell or otherwise be altered if exposed to oxygen and/or moisture. Therefore, packaging that includes a suitable barrier to moisture and/or oxygen may improve the storage conditions for a medical device and may improve the shelf life of the device.

Current solutions for controlling oxygen and/or water vapor in packaging headspace may include the use of high barrier film pouches to control ingress/egress of headspace gases that may degrade product performance. In addition to the use of high barrier films, other design elements can be added for further control. For example, sachets or packets including moisture scavenging materials and/or oxygen scavenging materials may be used to absorb moisture and/or oxygen and/or for gas flushing for control of oxygen. The challenge with the use of sachets or packets is that their effectiveness or useful life is impacted as soon as they are exposed to moisture or oxygen. For example, drying agents, such as desiccants, absorb moisture upon exposure to the atmosphere, which starts the utilization of their absorption capacity. Therefore, desiccants are packaged and shipped in protective packaging to preserve their moisture absorption capacity. Removal of the desiccants from their protective packaging may occur prior to placement of the desiccants into their final location. In some cases, the absorption capacity of desiccants may fall below the capacity required within the sealed pouch housing the medical device, after approximately one hour of exposure to the atmosphere.

In order to control the headspace moisture within the sealed pouch and preserve the useful life of the desiccant, either a two-pouch design including a sterile header bag inside a foil pouch or a complex sterile barrier pouch with two chambers may be used. A two-pouch design may require a larger foil pouch than a dual-chamber pouch and therefore a larger shelf carton. Additionally, a two-pouch design may require the user to open two separate pouches to access the product. In some cases, the desiccant in a two-pouch design may not be sterile or the location may not be controlled within the foil pouch. In such an instance, the desiccant may contaminate the sterile field. In a two-chamber design, one chamber of the two-chamber pouch may be sterile while the other is not. After the sterilization process, the desiccant may be inserted into the non-sterile chamber of the packaging system. This may manage the desiccant's position within the pouch as well as initiate the activation of the desiccant due to exposure to the atmosphere. However, two-chamber pouches may be difficult to manufacture. The increased complexity of the two-chamber pouches may increase the risk of quality defects. Further, two-chamber pouches may be more expensive to manufacture than two-pouch designs and/or have a restricted supply.

Disclosed herein are a number of medical device packages and/or packaging components, methods for packaging and/or preparing a medical device for sale, and methods for improving the shelf life of medical devices. In some embodiments, the desiccant may be inserted into the sterile barrier to preserve the capacity of the desiccant. This may be accomplished through the design of the desiccant packaging and controlling when absorption begins inside the sterile barrier. In some embodiments, the desiccant may be placed inside the sealed sterile barrier with the medical device prior to sterilization, preventing premature activation and allowing the desiccant to maintain sufficient absorption capacity for the life of the medical device. Further, the packaging systems described herein may allow for a less complex single chamber sterile barrier pouch. In some cases, the packaging systems described herein may control a position of the desiccant sachet to allow for a more controlled aseptic opening of the packaging and removal of the medical device. Other features and benefits are also disclosed, as disclosed herein. While the devices and systems disclosed herein are described with respect to a desiccant, the devices and/or systems may additionally or alternatively include oxygen scavengers or oxygen absorbers, as desired.

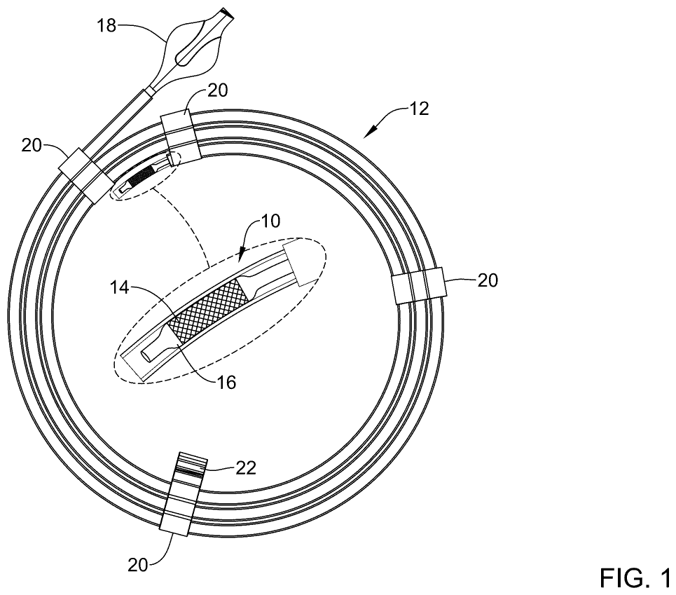

FIG. 1 illustrates a side view of an example medical device 10 disposed within a carrier tube 12. In this example, the medical device 10 may include a catheter for delivery of an endoprosthesis. For example, the medical device 10 may include an endoprosthesis or stent 14 attached thereto. In some embodiments, the stent 14 may be disposed on a balloon 16, which may be used to expand the stent 14. In other embodiments, the stent 14 is a self-expanding stent and, as such, the medical device 10 may be a catheter suitable for delivery thereof. In either embodiment, the stent 14 may be a bare metal stent, a coated stent, and/or may include one or more substances associated therewith such as pharmaceuticals or the like (e.g., the stent 14 may be a drug-coated or drug-eluting stent). The medical device 10 may also include a proximal hub 18. It can be appreciated that the medical device 10 may differ vastly in form and can include any other suitable medical devices including catheters, guidewires, or the like.

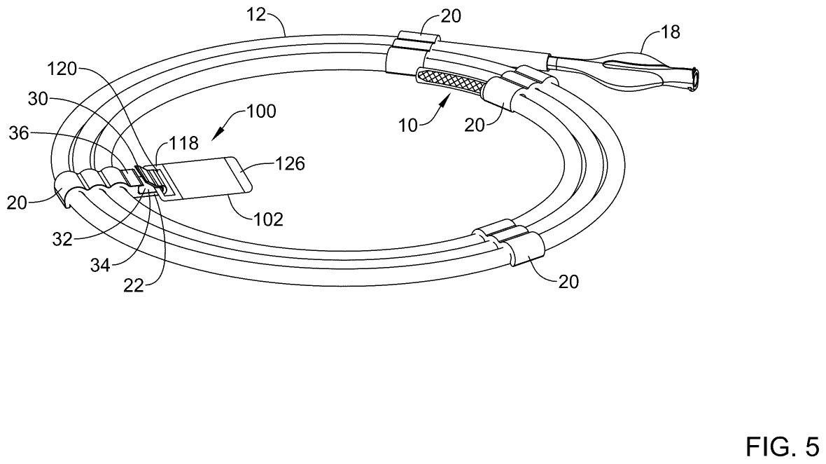

The carrier tube 12 may generally be configured to hold the medical device 10 in a suitable configuration. In at least some embodiments, the carrier tube 12 may be arranged or otherwise configured as a coil that allows the medical device 10, which may have a reasonably long length, to be held in a compact configuration. The individual windings of the coil may be secured together by one or more clips 20 configured to hold the carrier tube 12 in a predetermined configuration as is typical in the art. Other configurations for the carrier tube 12 include configurations suitable for holding other medical devices. At least one of the clips 20 may include a label retention clip 22. The label retention clip 22 may be configured to couple to a compliance chart (not explicitly shown) and/or a desiccant sachet or packet (see, for example, FIG. 5), as will be described in more detail here.

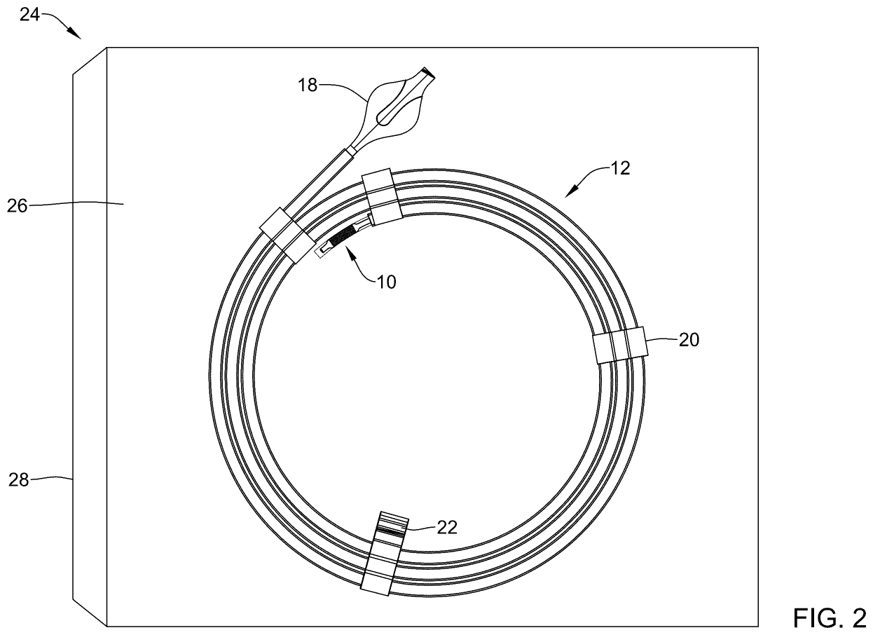

FIG. 2 is a schematic view of the medical device 10 disposed within an interior of a medical device package or packaging assembly 24. In general, the package 24 may take the form of an external packaging 24 including a flexible pouch, a rigid tray, a clamshell tray, or the like. An illustrative external packaging 24 may include a pouch that includes a first or “front” portion 26 and a second or “back” portion 28. In some embodiments, at least one of first portion 26 and the second portion 28 is substantially transparent so as to allow a clinician to see or view the medical device 10 while the medical device 10 is within the package 24. However, this is not required. In some cases, at least a portion of the package 24 may be opaque. In some examples, the package may be a sealed clear film/foil chevron pouch with a TYVEK® footer to enable sterilization and gas flushing.

In some embodiments, the package 24 may be considered a “primary” pouch in that the medical device 10 may be disposed directly within the package 24. It is contemplated that the package 24 may be the entire structure between medical device 10 and the exterior environment. In some embodiments, the first and second portions 26, 28 may be formed of a layer or film of material, which may or may not be the same material. Alternatively, the first portion 26 and/or the second portion 28 may include a plurality of layers. For example, the first and/or second portions 26, 28 may include two, three, four, five, six, seven, eight, nine, ten, eleven, twelve, or more layers. The number and/or make-up of the layers may or may not be the same for the first portion 26 and the second portion 28 and can vary. An illustrative multi-layer package is described in commonly assigned U.S. Pat. No. 9,096,368, titled MEDICAL DEVICE PACKAGING AND METHODS FOR PREPARING AND PACKAGING MEDICAL DEVICES, the disclosure of which is hereby incorporated by reference. Some illustrative materials that may be used to form the first and/or second portions 26, 28 may include, but are not limited to, aluminum foil, high-density polyethylene, medium-density polyethylene, low-density polyethylene, linear low-density polyethylene, ethylene vinyl acetate (EVA) and/or copolymers thereof, ethylene methacrylate (EMA), ethylene acrylic acid (EAA), ethylene methacrylic acid (EMAA), ethylene acrylates, polyethylene terephthalate, coated polyethylene terephthalate film, blends and/or copolymers thereof, or the like. These are just examples. Other polymers are contemplated including any of those disclosed herein.

In some embodiments, the first and/or second portions 26, 28 may include a moisture scavenging material, for example a chemical desiccant, such a portion of the package may be considered to be moisture scavenging layer. The chemical desiccant may be any suitable material such as calcium oxide, magnesium oxide, barium oxide, barium hydroxide, strontium oxide, aluminum oxide, partially hydrated aluminum oxide, magnesium sulfate, sodium phosphate di-basic, ammonium chloride, potassium carbonate, potassium aluminum disulfate, magnesium chloride, diammonium sulfate, sodium nitrate, calcium chloride, calcium sulfate, sodium chloride, potassium bromide, molecular sieves, clays and blends of these materials.

In some embodiments, the first and/or second portions 26, 28 may include an oxygen scavenging component, which may be similar to those available from CRYOVAC® (including, for example, material and/or films such as CRYOVAC® oxygen scavenging film OS2000). The oxygen scavenging component or film may be blended with a poly(ethylene-co-vinyl alcohol) and may be activated or pre-activated by exposure to ultraviolet light. The oxygen scavenging component may remove residual headspace oxygen within package 24 and, for example, add further protection from environmental oxygen.

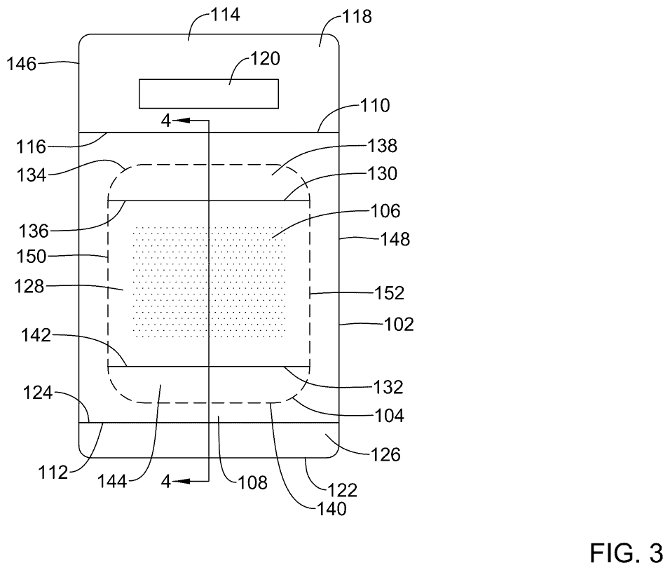

FIG. 3 is a schematic top view of an illustrative environmental control packet 100 such as a moisture or oxygen scavenging packet 100 for enclosing a desiccant or oxygen scavenging material. The packet 100 may include an outer porous barrier pouch 102, an inner non-porous container 104 and a desiccant 106. While the packet 100 is described as enclosing a desiccant, it should be understood that the packet 100 may additionally or alternatively enclose an oxygen scavenging material. The desiccant may be any suitable material such as calcium oxide, magnesium oxide, barium oxide, barium hydroxide, strontium oxide, aluminum oxide, partially hydrated aluminum oxide, magnesium sulfate, sodium phosphate di-basic, ammonium chloride, potassium carbonate, potassium aluminum disulfate, magnesium chloride, diammonium sulfate, sodium nitrate, calcium chloride, calcium sulfate, sodium chloride, potassium bromide, molecular sieves, clays and blends of these materials. Generally, the packet 100 may be configured to control when the desiccant 106 begins to absorb moisture within the sterile barrier of the package 24. Further, as will be described in more detail herein, the packet 100 may be configured to be placed directly within a single chamber sterile barrier which may mitigate some of the problems of the two-pouch package design and/or the dual chamber pouch design described herein. While the packet 100 is described with respect to use in a single chamber flexible pouch, it should be understood that the packet 100 may be used with other medical device packaging systems, including, but not limited to, two-pouch packages, dual chamber pouches, clamshell trays, rigid trays, cardboard backing cards with sterile plastic wrap, and the like.

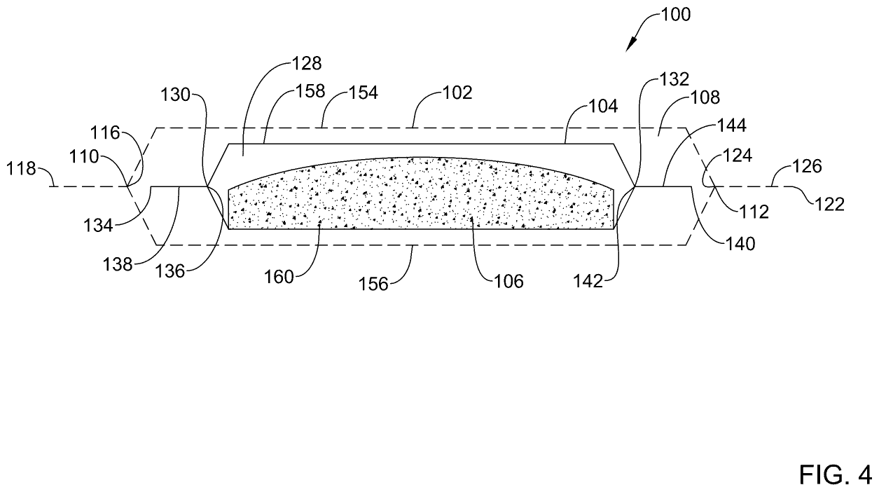

The outer barrier pouch 102 may be formed from a porous material such as, but not limited to, a high-density polyethylene (e.g., TYVEK®). The outer barrier pouch 102 may define an inner cavity 108 extending between a first seal 110 and a second seal 112 and configured to receive the inner sachet 104. Sealing the inner container 104 within the outer barrier pouch 102 may generally include disposing the inner container 104 within the outer barrier pouch 102 in which three sides of a first portion 154 and a second portion 156 (see, for example, FIG. 4) of the outer barrier pouch 102 are sealed together so as to form the outer barrier pouch 102 into a pouch. After inserting the inner container 104 into the outer barrier pouch 102, the final side may be sealed. It is contemplated that the outer barrier pouch 102 may be made using standard pouch/bag filling and scaling equipment.

The first and second seals 110, 112 may extend from a first lateral side 146 to a second lateral side 148 to create a fully enclosed inner cavity 108. For example, the inner cavity 108 may be sealed about an entirety of the perimeter thereof. In some examples, the first seal 110 may extend from a first end 114 of the outer barrier pouch 102 to a first end 116 of the inner cavity 108 to define a header region 118. The header region 118 may be a region where two or more layers (e.g., a front side 154 and a back side 156) of the outer barrier pouch 102 are sealed together. In some embodiments, the header region 118 may include an aperture or slot 120 extending through the thickness thereof. The slot/slit 120 may have a length less that a width of the outer barrier pouch. As will be described in more detail herein, the label retention clip 22 may be positioned within the slot/slit 120 to secure the packet 100 to the medical device 10 in a known and predictable location. In some cases, the slot 120 may be replaced with a narrow opening such as a slit or other opening extending through a thickness of the header region 118. In some examples, the second seal 112 may extend from a second end 122 of the outer barrier pouch 102 to a second end 124 of the inner cavity 108 to define a sealed region 126. Similar to the header region 118, the sealed region 126 may be a region where two or more layers (e.g., a front side 154 and a back side 156) of the outer barrier pouch 102 are sealed together. In some embodiments, the header region 118 may be longer than the sealed region 126. However, this is not required. In other examples, the header region 118 and the sealed region 126 may be approximately the same length or the sealed region 126 may be longer than the header region 118. While not explicitly shown, in some cases, the outer barrier pouch 102 may include one or more additional seals extending from the first end 114 to the second end 122 thereof. In some cases, the one or more additional seals may extend along the first and/or second lateral sides 146, 148, although this is not required.

The inner container 104 may be disposed within the cavity 108 of the outer barrier pouch 102. In the illustrated embodiments, the inner container 104 may be a sachet. However, the inner container 104 may be a more rigid container including, but not limited to, a vial or ampule. The inner sachet 104 may be disposed between the first seal 110 and the second seal 112 of the outer barrier pouch 102. The inner sachet 104 may be formed from a non-porous material, such as, but not limited to a polymer film having a very low moisture transmission rate which may minimize the ingress of moisture to the desiccant 106 and protect the longevity of the desiccant 106 disposed therein. It is contemplated that the inner sachet 104 may be made using standard pouch/bag filling and sealing equipment. The inner sachet 104 may define an inner cavity 128 extending between a first seal 130 and a second seal 132 and configured to receive the desiccant 106. Sealing the desiccant 106 within the inner sachet 104 may generally include disposing the desiccant 106 within the inner sachet 104 in which three sides of a first portion 158 and a second portion 160 (see, for example, FIG. 4) of the inner sachet 104 are sealed together (so as to form the inner sachet 104 into a pouch. After inserting the desiccant 106 into the inner sachet 104, the final side may be sealed.

The first and second seals 130, 132 may extend from a first lateral side 150 to a second lateral side 152 to create a fully enclosed inner cavity 128. For example, the inner cavity 128 may be sealed or otherwise closed about an entirety of the perimeter thereof. This may encapsulate the desiccant 106 within the cavity 128 and prevent the desiccant 128 from exiting the cavity 128. In some examples, the first seal 130 may extend from a first end 134 of the inner sachet 104 to a first end 136 of the inner cavity 128 to define a first sealed region 138. The first sealed region 138 may be a region where two or more layers (e.g., a front side 158 and a back side 160) of the inner sachet 104 are sealed together. In some examples, the second seal 132 may extend from a second end 140 of the inner sachet 104 to a second end 142 of the inner cavity 128 to define a second sealed region 144. Similar to the first sealed region 138, the second sealed region 144 may be a region where two or more layers (e.g., a front side 158 and a back side 160) of the inner sachet 104 are sealed together. In some embodiments, the first and second sealed regions 138, 144 may be approximately the same length. However, this is not required. In other examples, the first and/or second sealed regions 138, 144 may have differing lengths. While not explicitly shown, in some cases, the inner sachet 104 may include one or more additional seals extending from the first end 134 to the second end 140 thereof. In some cases, the one or more additional seals may extend along the first and/or second lateral sides 150, 152, although this is not required.

FIG. 4 is a partial cross-sectional view of the illustrative packet 100 of FIG. 3, taken at line 4-4 of FIG. 3. As can be seen in FIG. 4, the outer barrier pouch 102 may include a first portion 154 and a second portion 156 joined together at the first and second seals 110, 112. Between the first and second seals 110, 112, the first and second portions 154, 156 may be spaced from one another to define the inner cavity 108. Similarly, the inner sachet 104 may include a first portion 158 and a second portion 160 joined together at the first and second seals 130, 132. Between the first and second seals 130, 132, the first and second portions 158, 160 may be spaced from one another to define the inner cavity 128. Generally, the packet 100 may be configured to control when and how the desiccant 106 is exposed to atmosphere. For example, when the desiccant 106 is disposed within the inner sachet 104, the desiccant 106 may be substantially isolated from atmosphere or the moisture ingress through the inner sachet 104 minimized. The inner sachet 104 may be configured to selectively rupture or break to allow the desiccant 106 to be exposed to the cavity 108 of the outer barrier pouch 102. As the outer barrier pouch 102 is porous, once the desiccant 106 is exposed to the cavity 108, the desiccant 106 may be exposed to atmosphere and start absorbing moisture. It is contemplated that the material of the inner sachet 104 and/or the seals 130, 132 of the inner sachet 104 may be configured to fail after a sterilization procedure using a pressure differential, physical or mechanical means, or a combination thereof. When the inner sachet 104 is ruptured, burst, or otherwise opened, the inner sachet 104 remains sealed within the outer barrier pouch 102 which acts as a sterile barrier between the desiccant 106 and the interior of the package 24. Further, the outer barrier pouch 102 contains the exposed desiccant 106 while allowing the desiccant 106 to absorb moisture.

In some examples, the packet 100 may be releasably coupled with the label retention clip 22 maintaining the carrier tube 12 in a coiled configuration. However, this is not required. FIG. 5 is a schematic perspective view of the packet 100 coupled with the label retention clip 22. The label retention clip 22 may include a clasp 30 spaced a distance from a body 36 of the label retention clip 22 to define a gap 32. The slot/slit 120 of the header region 118 of the outer barrier pouch 102 may be slid within the gap 32 to position a portion of the header region 118 within a slot 34 of the label retention clip 22. The gap 32 may be large enough to allow the header region 118 to be easily coupled with the label retention clip 22 while precluding inadvertent uncoupling for the packet 100 from the label retention clip 22. It is contemplated that the packet 100 may be coupled with the label retention clip 22 prior to placing the medical device 10 within the package 24 and prior to sterilization of the medical device 10. Securing the packet 100 to the label retention clip 22 may position the packet 100 in an expected and predictable location such that the user knows where the packet 100 is prior to opening the package 24, even in cases where the package 24 may be opaque. When the packet 100 is secured to the label retention clip 22, the desiccant 106 may be enclosed within the inner sachet 104 thus preventing the desiccant 106 from being exposed to atmosphere as the medical device 10 is placed in the package 24. As such, as the packet 100 is being coupled with the label retention clip 22, the useful life of the desiccant 106 remains intact.

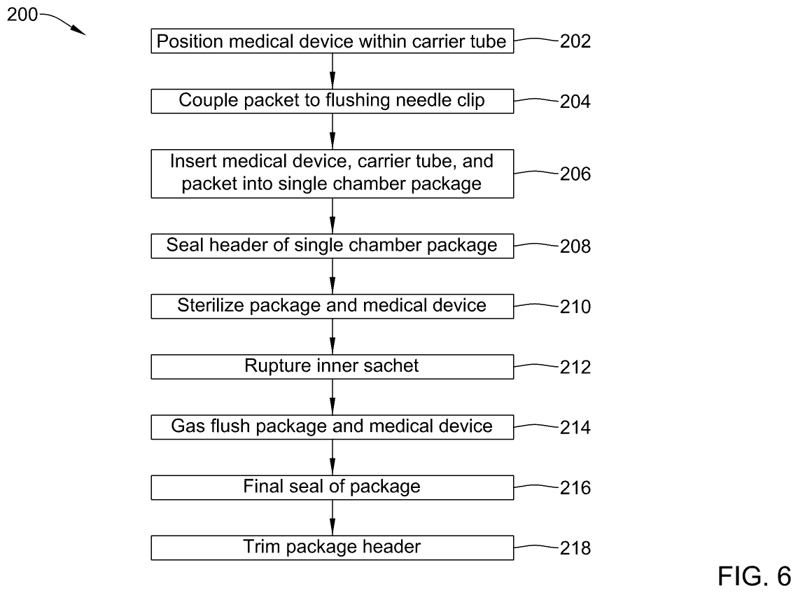

FIG. 6 is an illustrative flow chart 200 of a method for packaging the medical device 10 and activating the desiccant 106 to form a medical device packaging assembly. To begin, the medical device 10 may be secured within a carrier tube 12, or another carrier device appropriate for the particular medical device 10, as shown at block 202. The packet 100 may then be optionally coupled to the label retention clip 22, or other clasp member of the carrier tube 12, as shown at block 204. The medical device 10, carrier tube 12, and packet 100 may then be positioned within the chamber of an external package 24, as shown at block 206. It is contemplated that the package 24 may be a single chamber pouch. However, this is not required. In some cases, the package 24 may be a two-chamber pouch, a rigid tray, a clamshell tray, or the like. Once the medical device 10 and the packet 100 have been positioned within the package 24, the package 24 may be sealed, as shown at block 208. In some cases, the package 24 may be sealed at the header region. Sealing the medical device 10 within the package 24 may generally include disposing the medical device 10 and the packet 100 within the package 24 in which three sides of the first portion 26 and the second portion 28 of the package 24 are sealed together (e.g., heat sealed) so as to form the package 24 into a pouch. After inserting the medical device 10 into the package 24, the final side may be sealed (e.g., heat sealed). However, other sealing techniques may be used to suit the package type.

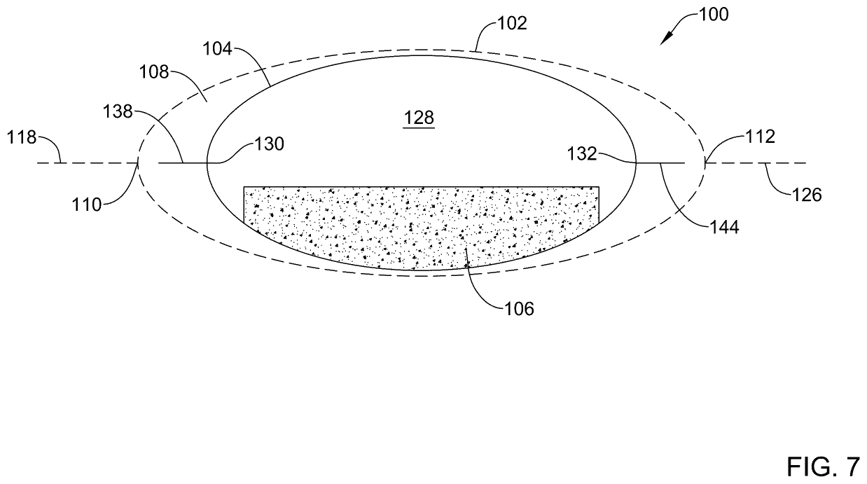

Next, the package 24 may be sterilized, as shown at block 210. Sterilization of the package 24 may sterilize the contents thereof including the medical device 10 and the packet 100. For example, the medical device 10, the packet 100, and/or the package 24 may be sterilized using any suitable sterilization procedure such as ethylene oxide sterilization, radiation, steam sterilizing, autoclaving, or the like. During the sterilization process, a vacuum may be pulled on the package 24. For example, the initial pressure at the time of sterilization may be about 1013 millibar (mbar) (101.3 kilopascals). The pressure may be reduced to a final pressure in the range of about 54-83 mbar (5.4-8.3 kilopascals) at a vacuum rate of 34 mbar per minute. The vacuum may be applied for in the range of about 28 to about 60 minutes. This is just one example of a suitable sterilization process. Other sterilization processes may be used as desired. For example, the vacuum rate may be more or less than 34 mbar per second and/or the final pressure may be more than 83 mbar or less than 54 mbar. Further the vacuum may be applied for less than 28 minutes or more than 60 minutes, as desired. As the packet 100 is in the package 24, the exterior surface of the outer barrier pouch 102 may undergo sterilization as well. Referring briefly to FIG. 7, which illustrates a schematic cross-sectional view of the packet 100 during the sterilization process, the outer barrier pouch 102 and inner sachet 104 may expand radially outwardly under the vacuum. However, the seals 110, 112 of the outer barrier pouch 102 and the seals 130, 132 of the inner sachet 104, as well as the polymer film of the inner sachet 104 may remain intact and fully sealed. Thus, the desiccant 106 remains encapsulated within the cavity 128 of the inner sachet 104 and substantially isolated from the atmosphere.

After the sterilization procedure, the inner sachet 104 may be ruptured or opened to form one or more openings 150a, 150b connecting the inner cavity 128 of the inner sachet 104 with the inner cavity 128 of the outer barrier pouch 102 to expose the desiccant 106 to the atmosphere, as shown at block 212. In some cases, the inner sachet 104 may be ruptured prior to or substantially simultaneously with a gas flushing procedure to ensure any oxygen inside of the inner sachet 104 is removed. In some cases, the inner sachet 104 may be ruptured by applying a physical or mechanical force to the packet 100 through the package 24 to burst or rupture the inner sachet 104. The physical or mechanical force may be applied by a user's hand, a manual tool, an automated tool, etc. The inner sachet 104 may include predefined areas of weakness such as, but not limited to, laser perforations or the seams 130, 132 along which the polymer film may open. It is contemplated that in some cases the inner container 104 may be formed from a rigid crushable ampule or vial (e.g., plastic or glass) allowing the inner container 104 to break in response to the applied force to expose the desiccant 106 therein. In other examples, ampules or vials may include a cap, plug, or other component releasably coupled thereto and configured to partially or fully release from the ampule or vial in response to a predetermined pressure differential thus exposing the desiccant 106 to the interior cavity 128 of the outer barrier pouch 102 and the atmosphere. In yet other examples, a sonic generator may be used to generate a frequency at which a rigid ampule, vial, or container may fail subsequently exposing the desiccant 106.

In some cases, ampules, vials, perforations, scoring, or the seams 130, 132 in the inner sachet 104 may allow the inner sachet 104 to fail at a predetermined pressure. For example, the inner sachet 104 may be ruptured through internal and external pressure differentials of the inner sachet 104 by increasing or decreasing the air pressure around the inner sachet 104 until it bursts. When the inner sachet 104 is configured to rupture at predetermined pressures, the rupturing of the inner sachet 104 may be achieved with repeatable results using an automated clamping system to apply a set pressure and distance of travel.

In another example, the perforations, scoring, or the seams 130, 132 in the inner sachet 104 may allow the inner sachet 104 to fail at a predetermined temperature. In such an instance, the inner sachet 104 may be ruptured or fail during the sterilization process or during a specific temperature-controlled process (e.g., oven exposure, etc.).

In yet another example, the inner sachet 104 may include a magnetic material disposed on or embedded within the polymer film. When it is desired to open the inner sachet 104, a magnetic field may be applied to the packet 100 causing the magnetic material to move towards the magnetic field. The magnetic material may apply a force to the inner sachet 104 which tears the polymer film and exposes the desiccant 106.

Additionally, it may be desirable to modify and/or control the atmosphere within the package 24. Thus, after sterilization, the package 24, medical device 10, and/or packet 100 may be gas flushed, as shown at block 214. This may include exposure to nitrogen, oxygen, water (e.g., moisture, humidity, etc.), argon or another inert atmosphere, other atmospheres, or the like. Exposure may include one or more cycles of exposure. If multiple cycles are desired, the cycles may have a vacuum exposure between cycles. During the gas flushing process, a vacuum may be pulled on the package 24. For example, the initial pressure at the time of gas flushing may be about 1013 millibar (mbar) (101.3 kilopascals). The pressure may be reduced to a final pressure in the range of about 5-29 mbar (0.5-2.9 kilopascals) at a vacuum rate of 328 mbar per second. The vacuum may be applied for in the range of about 0 to about 3 seconds. Thus, the gas flushing may undergo a larger pressure change over a shorter period of time relative to the sterilization process. This is just one example of a suitable gas flushing process. Other gas flushing processes may be used as desired. For example, the vacuum rate may be more or less than 328 mbar per second and/or the final pressure may be more than 29 mbar or less than 5 mbar. Further the vacuum may be applied for more than 3 seconds, as desired. As noted above, in some cases, the inner sachet 104 may be ruptured prior to the gas flushing procedure. In other cases, the inner sachet 104 may be ruptured during the gas flushing procedure.

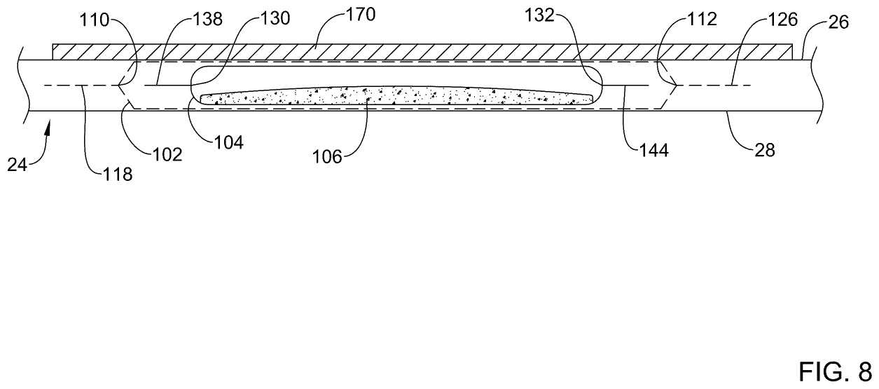

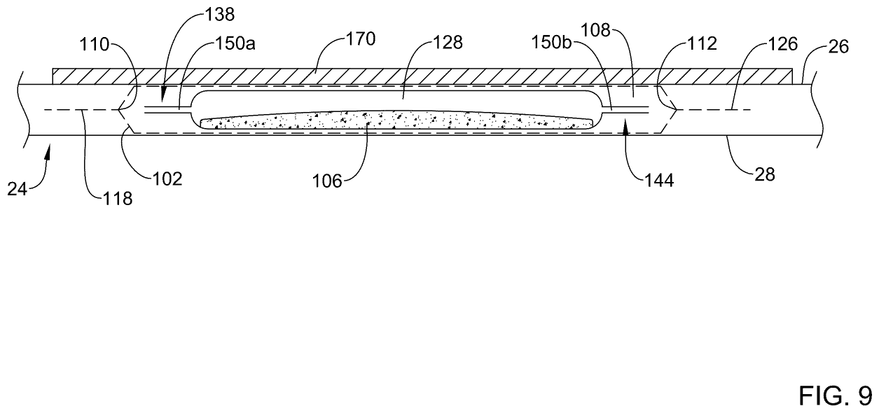

Referring briefly to FIG. 8, which illustrates a schematic cross-sectional view of the packet 100 during an illustrative gas flushing process, in some cases, a restrictor plate 170 may be positioned over the packet 100 exterior to the package 24. The restrictor plate 170 may prevent the outer barrier pouch 102 and/or inner sachet 104 from expanding radially outwardly during the gas flushing process. For example, the restrictor plate 170 may be used during the gas flushing process to reduce the volume of the packet 100 and to increase the strain on the film or seals 130, 132 of the inner sachet 104 to a greater degree than experienced during sterilization causing the seals 130, 132 to fail or burst, as shown in FIG. 9, which illustrates a schematic cross-sectional view of the packet 100 after the gas flushing process and/or after rupture of the inner sachet 104. In some cases, the inner sachet 104 may be provided with laser scores, perforations, or other weakened regions configured to cause the inner sachet 104 to open and/or fail at a predetermined location and at a predetermined rate of pressure change and or pressure differential. It is further contemplated that the inner sachet 104 may be configured to burst with an ultimate yield at a higher strain rate and/or pressure differential than can be achieved during sterilization. While the gas flushing process is described as including a restrictor plate 170 to limit and/or control the expansion of the inner sachet 104, in some cases, the inner sachet 104 may open and/or fail using the gas flushing process alone (e.g., with no physical restriction to the inner sachet 104). As described above, the inner sachet 104 is disposed within the cavity 108 of the outer barrier pouch 102. The outer barrier pouch 102 may maintain control of the inner sachet 104 and act as a sterile barrier between the desiccant 106 and medical device 10. For example, the outer barrier pouch 102 may ensure microbes from the inner sachet 104 and the desiccant 106 do not migrate inside of the sterile package 24. Once the inner sachet 104 has been opened, the desiccant 106 is exposed to the interior cavity 108 of the outer barrier pouch 102 and can begin absorbing moisture through the permeable outer barrier pouch 102. The material and/or the seals 110, 112 of the outer barrier pouch 102 may be configured to withstand the strain and/or pressure differential during the gas flushing process such that the outer barrier pouch 102 remains fully sealed to enclose the inner sachet 104 and desiccant 106 after the inner sachet 104 has burst or opened. In some embodiments, the outer barrier pouch 102 may be formed from a transparent or clear material which allows for visual confirmation that the inner sachet 104 has opened.

Returning to FIG. 6, after the gas flushing procedure, the package 24 may be sealed again at the header region, as shown at block 216. Finally, extra header material may be trimmed from the package 24, as shown at block 218. Positioning the packet 100 within the package 24 places the desiccant 106 inside the sealed sterile barrier with the medical device 10 prior to sterilization while preventing activation of the desiccant 106 and allowing the desiccant 106 to maintain sufficient absorption capacity for the life of the medical device 10. Further, the packet 100 may allow for control of the absorption capacity of the desiccant 106 while using a single chamber sterile barrier pouch.

It is further contemplated that positioning the packet 100 within a single chamber pouch package 24 may allow for a simpler pouch design which may be easier to manufacture and may be more broadly available for purchase. In some cases, a single chamber pouch may be less expensive to manufacture or purchase than a dual chamber pouch. For example, in some cases, a single chamber pouch may be about one third the cost of a dual chamber pouch. It is further contemplated that positioning the packet 100 in a controlled position (e.g., at the label retention clip 22) may allow the user may maintain sterility of the medical device 10 while opening only a single chamber pouch. Further, as the packet 100 was in the sterile barrier of the package 24 (e.g., the packet 100 was sterilized) there may be less risk of contaminating the medical device 10 with the packet 100.

It is contemplated that other activation techniques may be used to open or burst the inner sachet 104. In one illustrative example, the inner sachet 104 may be formed at least in part from a degradable polymer which allows the inner sachet 104 to fail during a gas flushing pressure differential. This may be achieved by using a film structure, such as polylactic acid (PLA), which may begin to degrade during the sterilization process. The PLA in the inner sachet 104 may degrade during the sterilization process to the point at which the inner sachet 104 is weakened and would fail during the gas flushing process. In another example, the inner sachet 104 may be configured to degrade during radiation sterilization. For example, the inner sachet 104 may fail during radiation sterilization or during the gas flushing process.

In yet another example, an alternative method of controlling the moisture inside the sterile barrier (e.g., the package 24) may include a desiccant 106 with a controlled rate of absorption which may prevent the complete consumption of the desiccants capacity when exposed to the atmosphere and sterilization prior to the final sealing of the sterile barrier. Controlling the rate of absorption may be accomplished by encapsulating the desiccant material 106 inside a polymer(s) with a predetermined moisture transmission rate. For example, the polymer may encapsulate individual beads of the desiccant 106 and the inner sachet 104 may be omitted. The transmission rate of the polymer(s) controls the rate of moisture ingress to the desiccant 106, thereby controlling the rate at which the desiccant's 106 absorbing capacity is consumed. Controlling the desiccant's 106 rate of absorption, may allow for the desiccant 106 to be placed in the porous sterile barrier 102 and be exposed to the atmosphere and sterilization, while maintaining sufficient desiccant absorption capacity for the life of the medical device 10. Alternatively, or additionally, a bio-degradable polymer such as, but not be limited to, PLA could be used to coat the surface of the desiccant 106 and over a period of time the bio-degradable polymer may breakdown exposing the desiccant 106. In some examples, heat may be used to initiate or accelerate the breakdown of a bio-degradable polymer. In some cases, complete degradation of PLA can be achieved in as little as 100 hours at 37° C.

While a number of materials are listed above for use with the package 24, outer barrier pouch 102, and/or inner sachet 104, other polymeric materials are also contemplated. Some examples of suitable polymers include polytetrafluoroethylene (PTFE), ethylene tetrafluoroethylene (ETFE), fluorinated ethylene propylene (FEP), polyoxymethylene (POM), for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate (EVA) and/or copolymers thereof, ethylene methacrylate (EMA), ethylene acrylic acid (EAA), ethylene methacrylic acid (EMAA), silicones, polyethylene (PE), high-density polyethylene (for example MARLEX® high-density polyethylene), medium-density polyethylene, low-density polyethylene (for example MARLEX® low-density polyethylene), linear low density polyethylene (for example REXELL®), ionomer, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device packaging assembly, comprising:
an external package;
a carrier tube disposed within the external package, the carrier tube including one or more clips configured to hold the carrier tube in a predetermined configuration;
a medical device disposed within the carrier tube; and
a moisture or oxygen scavenging packet releasably coupled to one of the one or more clips, the moisture or oxygen scavenging packet comprising:
an outer barrier pouch;
an inner container disposed within the outer barrier pouch; and
a desiccant and/or an oxygen scavenging material disposed within the inner container.

2. The medical device packaging assembly of claim 1, wherein the inner container comprises a non-porous material.

3. The medical device packaging assembly of claim 1, wherein the outer barrier pouch comprises a porous material.

4. The medical device packaging assembly of claim 1, wherein the inner container is configured to rupture in response to an applied force.

5. The medical device packaging assembly of claim 1, wherein the inner container comprises a sachet formed from a polymer film.

6. The medical device packaging assembly of claim 1, wherein the inner container comprises a crushable vial.

7. The medical device packaging assembly of claim 1, wherein the outer barrier pouch comprises a sealed header region.

8. The medical device packaging assembly of claim 7, further comprising an opening extending through a thickness of the sealed header region.

9. A method for packaging a medical device, the method comprising:
coupling an environmental control packet to a clip secured relative to a medical device, the environmental control packet comprising:
an outer barrier pouch;
an inner container disposed within the outer barrier pouch; and
a desiccant and/or an oxygen scavenging material disposed within the inner container;
disposing the medical device, clip, and environmental control packet within an external package;
sealing the external package;
sterilizing the external package;
rupturing the inner container; and
gas flushing the external package.

10. The method of claim 9, wherein rupturing the inner container and gas flushing the external package occur substantially simultaneously.

11. The method of claim 9, further comprising positioning a restrictor plate over the environmental control packet prior to gas flushing the external package.

12. The method of claim 9, wherein rupturing the inner container comprises applying a physical force to the inner container.

13. The method of claim 9, wherein rupturing the inner container comprises applying a pressure differential to the inner container.

14. The method of claim 9, wherein rupturing the inner container comprises exposing the inner container to a predetermined temperature.

15. The method of claim 9, wherein rupturing the inner container activates a useful life of the desiccant and/or oxygen scavenging material.

16. An environmental control packet for use with a medical device packaging assembly, the environmental control packet comprising:
a porous outer barrier pouch;
a non-porous inner container disposed within the outer barrier pouch; and
a desiccant and/or an oxygen scavenging material disposed within the inner container;
wherein the inner container is configured to rupture in response to an applied force.

17. The environmental control packet of claim 16, wherein the inner container comprises a sachet formed from a polymer film.

18. The environmental control packet of claim 16, wherein the inner container comprises a crushable vial.

19. The environmental control packet of claim 16, wherein the outer barrier pouch comprises a sealed header region.

20. The environmental control packet of claim 19, further comprising an opening extending through a thickness of the sealed header region.
